(19) United States
(12) Patent Application Publication
(10) Pub. No.: US 2008/0268431 A1
(43) Pub. Date: Oct. 30, 2008

(54) Information Code System Using Dna Sequences

(75) Inventors: Jin-Ho Choy, Seoul (KR); Man Park, Daegu-city (KR); Jae-Min Oh, Seoul (KR)

Correspondence Address: ROTHWELL, FIGG, ERNST & MANBECK, P.C., 1425 K STREET, N.W., SUITE 800, WASHINGTON, DC, 20005, US

(73) Assignee: Jin-Ho CHOY, Seoul (KR)

(21) Appl. No.: 11/575,252

(22) PCT Filed: Sep. 14, 2004
(86) PCT No.: PCT/KR2004/002329

§ 371 Date: Mar. 14, 2007

(51) Int. Cl. C12Q 1/68; C07H 21/00

(52) U.S. Cl. 435/6; 536/23.1

(57) ABSTRACT

The present invention provides a molecular level of DNA information code which uses a base pair sequence as an information code unit. Also, the present invention provides a molecular code system which includes designing and coding DNA which is an information code unit; stabilizing the DNA information code by encapsulating it with an inorganic capsule and coating the DNA-inorganic capsule to a medium; taking and extracting the coated DNA information code which is present in a trace amount, collecting the DNA information code using a polypyrrole-maghemite nanohybrid; and amplifying the collected DNA information code using a polymerase chain reaction and reading the amplified DNA information code. According to the present invention, the DNA information code having high security is prepared by assigning a security unit to a DNA which has an excellent accumulating capacity, and then the DNA information code is stabilized so as to be coated to a medium. Only the DNA information code may be extracted, collected, and read, if necessary. Thus, a unified molecular code system can be established.

### CROSS REFERENCE TO RELATED APPLICATION(S)

This application is a 35 U.S.C. § 371 National Phase Entry Application from PCT/KR2004/002329, filed Sep. 14, 2004, and designating the United States.

### BACKGROUND OF THE INVENTION

1. Field of the Invention

The present invention relates to a molecular level of information code using a DNA base sequence as an information unit, and more particularly, to a DNA information code comprising an information code region containing a specific base sequence, primer regions located on both ends, which are necessary to amplify the DNA and to read the DNA base sequence, and buffer regions, each buffer region being interposed between the information code region and the primer region and separating the information code region from the primer region by at least one nucleotide

2. Description of the Related Art

In general, a barcode is one of the most frequently used code systems. A barcode refers to a combination of letters or numbers in black or white bar-shaped symbols. Barcodes are used for rapid inputting and reading data. They are used in various applications, such as discrimination of various items, information management of sales, book classification, identity certification, etc. according to the Universal Product Code (UPC). In addition, barcode is a watermark or a code system to discriminate a bill from forgery. Such a code system is formed in four steps of information theory, i.e., acquiring and recording information, storing the recorded information, collecting and reading the information, and displaying the read information. A serious problem in using the code system is that it may be difficult to discriminate and verify whether an important document, an expensive item, or an identity card, etc, is authentic or forgery when it is copied or damaged. In practical, cases of counterfeit money have occurred due to copying of watermarks and may result in serious social and economical problems.

To overcome these problems, there is a need to effectively conceal a given code and protect the code against illegal copy. The effective concealment of the code and the copying protection can be accomplished by developing a code at a molecular level. The code has a fine size to be invisible to bare eyes, and cannot be easily detected when it is uniformly supported by a medium. Even duplication is impossible when it is modified using a special apparatus. The most suitable example of the molecular level of code is one using a DNA base pair as a code unit. All the genetic information of an organism is contained in a DNA and the DNA base pair can efficiently store the information. In addition, genetic information has inherent properties according to an individual, and thus, much research has been conducted on using the DNA as a code for discriminating the individual or classifying a kind.

Korean Patent Application No. 2001-0034002 having the title of “Coding method for discriminating kinds” describes classifying kinds of plants using DNA analysis. Korean Patent Application No. 1993-030237 having the title of “Method of discriminating species of Korean Bulls using DNA polymorphism analysis” describes using DNA like barcodes so as to discriminate whether the animals are authentic or forgery. Korean Patent Application No. 2000-0057825 having the title of “Means and methods for discriminating individuals using genes” suggests discriminating individuals by analyzing the genes of respective individuals and matching them to barcodes to designate an inherent barcode to the medium. International Patent Application No. WO 03/052101 having the title of “Sample tracking using molecular barcode” describes using arbitrarily prepared molecular codes (DNA, RNA, etc.) for discriminating samples or specimens which participate in biochemical reactions, rather than using the genomic DNAs of the individual, as described above.

Although the above research suggested using DNA as a molecular level of code or a barcode for classification, the DNA has a limited range of application. Since DNA can be easily destroyed or denatured when they are exposed to various factors, such as enzymatic environments, chemical and physical environments, etc., the use of DNA as a common code is limited unless the means for stabilizing the DNA are used. In addition, in order that the DNA is practically used as a code system, such as a barcode, there is a need for great advancements in the analytical methods of DNA. For a practical use, there is a need for methods which can analyze a trace amount of molecular codes according to the conditions, not using a large amount of molecular code. A representative method of efficiently collecting biomolecules, such as DNA, includes using magnetic particles, as suggested in International Published Application No. WO 95/11839. Although DNA can be easily collected using the magnetic particles in this method, a trace amount of DNA cannot be detected.

Thus, to establish a molecular code system having a wide range of application, there is a need for a method of stabilizing DNA against environmental factors and a method of detecting and collecting a trace amount of DNA. For this, conventional methods of manipulating DNA must be complemented and improved.

A molecular level of information code according to an embodiment of the present invention comprises DNA as a basic unit, like the conventional methods, but further comprises various security units and safety units, which are added when designing the DNA. Today's DNA manipulation methods can synthesize DNA of any combination and manipulate any base pair sequence at one's own ends, and thus, information can be coded and protected using a specific unit.

DNA may be stabilized using a capsule, for example, made of inorganic materials. The DNA stabilized using the inorganic materials may be effectively protected from enzymes, such as DNase and may be stabilized against chemical conditions, for example, acidic or basic conditions. Also, the DNA stabilized using the inorganic materials may be extracted with its information preserved, using a suitable chemical method.

Since the extracted DNAs are present in a diluent solution, it may be difficult to collect and read the information from the DNA. In general, a polymerase chain reaction (PCR) is used for detecting the information from the diluted DNA, which was developed by K. Mullis in the mid 1980s and was an innovative technique in the field of molecular genetics for researching and analyzing genes. In the PCR, the copy number of a specific DNA sequence can be exponentially increased. The PCR adopts DNA replication by DNA polymerase. In the PCR, DNA polymerase synthesizes a complementary DNA using a single-stranded DNA as a template. Such a single-stranded DNA can be obtained in a simple manner by denaturing double-stranded DNA. To start the DNA synthesis by DNA polymerase, the initiating portion of the strand is divided into two strands. When primers which can complementarily bind to both the ends of the DNA sequence to be amplified are added to the reaction, the primers bind to the both ends, thus initiating DNA synthesis. After the binding (annealing), the DNA is synthesized along the strand and extended to the opposite end of the strand by the action of polymerase.

As described above, a cycle of the PCR is composed of (1) denaturation, (2) annealing, and (3) extension. In the next cycle, the DNA, which was synthesized with the initial DNA in a previous cycle, is divided into two single-stranded DNA templates. Accordingly, in theory, the number of double-stranded DNA is 2n after n cycles. These amplified segments of DNA are subjected to gel electrophoresis and the presence of the DNA may be detected by confirming the specific band on the gel.

When the DNA is amplified using the PCR and the amplified DNA is confirmed using electrophoresis, the DNAs can be detected only at a predetermined level or higher in the sample. A trace amount of DNA cannot be effectively amplified using the PCR nor can the DNA be detected.

Generally, iron oxides are classified as goethite, lepidocrocite, hematite, magnetite, and maghemite, etc. depending on their structures. The magnetic properties of iron oxides can vary depending on their structures and in some cases, on the particle size. Iron oxide of the maghemite structure is advantageous in view of the magnetic properties, the structural stability, and the efficiency of synthesis, etc. Regarding the magnetic properties, the maghemite is ferromagnetic in a bulk state and superparamagnetic in nanoparticles having a size of 10 nm or less. Thus, the maghemite nanoparticles have an advantage in that they can maintain the superparamagnetic properties even when they are synthesized in the form of nanoparticles which have large specific surface areas.

Polypyrrole is well known as a conductive polymer. It is known that polypyrrole contains chloride ions, and thus, has an ability to adsorb various anions through a substitution reaction. Especially, a DNA, which has negatively charged phosphate groups, can be adsorbed to polypyrrole by the ion exchange reaction and the distances between the charges in polypyrrole are roughly similar to those between the negatively charged phosphate groups. Thus, polypyrrole adsorbs the DNA with high selectivity.

Thus, when the DNA is amplified using a method, such as the PCR, and then collected using a material, such as polypyrrole-iron oxide nanohybrid, a trace amount of the DNAs can be efficiently detected and easily read.

### SUMMARY OF THE INVENTION

In order to overcome the above problems, the present inventors conducted vigorous research and discovered that a DNA information code having specific information through a manipulation of a DNA base pair can be stabilized by encapsulating the DNA information code with an inorganic material and a trace amount of DNA can be selectively collected and then read using a functional nanohybrid, the nanohybrid being prepared by hybridizing maghemite nanoparticle, which has excellent magnetic properties, and polypyrrole, which has an excellent detection ability, at the nano level.

The present invention provides complete system including a DNA information code at a molecular level using a base pair as a basic information unit, a method of stabilizing the DNA information code by encapsulating the DNA information code with an inorganic material, a method of detecting a trace amount of DNA, which cannot be analyzed using conventional detection methods, using the characteristics of maghemite and polypyrrole, thus allowing the DNA information code to be read.

Thus, the present invention relates to the establishment of a molecular information code system including the preparation and stabilization of a molecular level of information code and collecting and reading of a trace amount of DNA.

According to an embodiment of the present invention, there is provided a DNA information code comprising an information code region containing a specific base sequence, primer regions located on both ends and which are necessary to amplify the DNA and read the DNA base sequence, and buffer regions whereby each buffer region is interposed between the information code region and the primer region and separates the information code region from the primer region by at least one nucleotide.

In the DNA information code, the base sequence of the information code region may be any base sequence, for example, CCT TAT ACG CTC AGT GTC, and preferably corresponds to a specific letter and/or number row, since when the base sequence is represented as a letter and/or number row which is a common data form, the information can be rapidly read.

In the DNA information code, the base sequence of the information code region may be coded by using the DNA base sequence itself as a code or by using the length of the base sequence as information. Each three-base pair may correspond to a letter and/or number. In this case, the base sequence may be expressed by letters, numbers and/or special letters in a total number of 64.

In the DNA information code, the sequence of the primer region must be kept confidential. In this case, the reading and amplification of the DNA cannot be performed, which is advantageous.

According to another embodiment of the present invention, there is provided a method of stabilizing a DNA information code, comprising:

preparing the above DNA information code; and

encapsulating the DNA information code with a layered double hydroxide.

In the stabilizing method, the layered double hydroxide encapsulating the DNA information code therein may be represented by the following formula:

[M²⁺₁₋ₓN³⁺ₓ(OH)₂][Aⁿ⁻]_{x/n}.yH₂O

wherein

M²⁺ is a divalent metal cation selected from the group consisting of Mg²⁺, Ni²⁺, Cu²⁺, and Zn²⁺,

N³⁺ is a trivalent metal cation selected from the group consisting of Al³⁺, Fe³⁺, V³⁺, Ti³⁺, and Ga³⁺,

x is a number of 0.1-0.4,

A is an anionic DNA,

n is a charge number of the DNA, and

y is a positive number.

In the stabilizing method, the layered double hydroxide may Ni2Al(OH)6(NO3), Zn2Al(OH)6(NO3), Mg2Fe(OH)6(NO3), Mg3Al(OH)8(NO3), etc., and preferably Mg2Al(OH)6(NO3). The layered compound has a cationic layer charge, and thus, may bind to DNA having negatively charged phosphate groups through electrostatic interaction.

In the stabilizing method, the layered double hydroxide may be synthesized using a conventional method which comprises preparing a solution of at least two divalent and trivalent metal salts and titrating the solution with a basic solution. It is preferable that the layered double hydroxide may be synthesized by titrating a 0.01-0.5 M aqueous solution in which magnesium nitrate and aluminum nitrate are mixed in a ratio of 1.5:1-2.5:1, in a nitrogen atmosphere with a 0.01-0.5 M sodium hydroxide solution until a pH of 9-10. If the numerical values are deviated from the above ranges, a compositional ratio of Mg to Al can be varied or impurities may be formed.

In the stabilizing method, the encapsulating may be performed using a conventional ion exchange reaction or a co-impregnation method. It is preferable that the encapsulating may be performed by dispersing the DNA information code and the layered double hydroxide in a molar ratio of 1:1-2:1 in decarbonated distilled water and stirring the obtained dispersion at 65-75° C. for 5-14 days in a nitrogen atmosphere. If the numerical values are not in the above ranges, the DNA base sequence may be modified or the stabilization of the DNA due to the layered double hydroxide may not be attained.

According to still another embodiment of the present invention, there is provided a DNA information code system which is stabilized using the above stabilization method.

The DNA information code system which is nano-sized, can be stably bound to any medium. The binding of the DNA information code system may be performed by dispersing the DNA information code in a solvent and then coating the resultant solution on a medium, or by directly incorporating the DNA information code into articles, for example, paper during its preparation, or by mixing the DNA information code into paints or coatings, etc.

According to yet another embodiment of the present invention, there is provided a method of detecting a specific DNA information code from any medium coated with the above DNA information code system, comprising:

taking a DNA-layered double hydroxide capsule from the medium;

extracting the DNA information code by dissolving the layered double hydroxide in a solvent;

collecting the extracted DNA information code using polypyrrole-maghemite hybrid nanoparticles;

amplifying the collected DNA information code using a PCR; and

reading the amplified DNA information code.

In the detecting method, the extraction of the DNA information code may be performed by dispersing the DNA-layered double hydroxide capsule in distilled water, adjusting the pH of the resultant dispersion to 2.5-3 by adding a phosphate buffer solution, and then stirring the dispersion for 20-40 minutes to dissolve the layered double hydroxide layer. If the numerical values are deviated from the above ranges, the DNA may not be efficiently extracted or the DNA may be damaged.

In the collecting the extracted DNA information code of the detecting method, the DNA information may be fully detected at a concentration of 500 femtomole (10-15 mol/L) or less, especially 100 femtomole or less.

In reading the amplified DNA information code of the detecting method, whether or not the amplified DNA information code is identical to a predetermined DNA information code may be determined by reading using electrophoresis, which can be performed easily and rapidly.

In the detecting method, the reading of the amplified DNA information code may be performed by sequencing the amplified DNA using an automatic sequencer and then converting the sequence to a corresponding letter and/or number row. In this case, the reading can be performed rapidly and conveniently.

According to a further embodiment of the present invention, there is provided a method of collecting DNA information code extracted from a DNA-layered double hydroxide using polypyrrole-maghemite hybrid nanoparticles.

In the collecting method, the polypyrrole-maghemite hybrid nanoparticles may be synthesized by dispersing maghemite nanoparticles in an excess of liquid pyrrole (a mass ratio of pyrrole/maghemite >0.7), removing an excess of pyrrole to obtain the maghemite nanoparticles with their surfaces wetted with pyrrole, adding an ethanol solution containing 0.1-0.2 M trivalent iron chloride to the wet maghemite nanoparticles and stirring for 0.5-1 hour to polymerize the pyrrole, and then, rinsing the resultant product with ethanol to remove an unreacted pyrrole therefrom. If the numerical values are deviated from the above ranges, the polymerization of pyrrole may not be easily performed or polypyrrole may not be uniformly applied to the maghemite particles.

In the collecting method, the polypyrrole-maghemite hybrid nanoparticles may be mixed with the extracted DNA solution to obtain a dispersion and then, polypyrrole-maghemite portions in the dispersion may be collected using a magnet. It is more preferable that 0.1 mg to several grams of the polypyrrole-maghemite hybrid nanoparticles is mixed with 10 μl to several ml's of a 100 fM-100 pM DNA solution and dispersed at 25-37° C. for 0.5-2 hours, and then, the polypyrrole-maghemite portions in the dispersion may be collected using a magnet. If the numerical values are deviated from the above ranges, the collection of the DNA using the polypyrrole-maghemite hybrid nanoparticles may not be fully completed.

### BRIEF DESCRIPTION OF THE DRAWINGS

The above and other features and advantages of the present invention will become more apparent by describing in detail exemplary embodiments thereof with reference to the attached drawings in which:

FIG. 1 is a flowchart illustrating a complete process according to an embodiment of the present invention;

FIG. 2 is a coding table for three-base pairs of DNA according to an embodiment of the present invention;

FIG. 3 is a schematic view illustrating a base pair sequence of the DNA information code according to an embodiment of the present invention;

FIG. 4 is a graph of X-ray diffraction of layered double hydroxide and DNA-layered double hydroxide capsule;

FIG. 5 is an electrophoresis result of DNA-layered double hydroxide nanohybrid treated with DNase I;

FIG. 6A is a transmission electron microscope photo of maghemite nanoparticles;

FIG. 6B is a transmission electron microscope photo of polypyrrole-maghemite nanohybrids;

FIG. 7 is an infrared (IR) spectrum for maghemite, polypyrrole-maghemite, and polypyrrole; and

FIG. 8 is an electrophoretic photo of DNAs which were collected using polypyrrole-maghemite hybrid nanoparticles and each of the 100 fM and 500 fM DNA solutions and amplified using a polymerase chain reaction (PCR).

### DETAILED DESCRIPTION OF THE INVENTION

Hereinafter, the present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art.

The DNA base pair sequence may be coded using various methods. In one of the most common methods, a three-base pair is used as a unit and each three-base pair corresponds to each of the Roman Alphabet letter and symbols (see FIG. 2), as the DNA codes genetic information. In addition, the DNA base pair sequence may be used as a code itself or coded by using the length of the base sequence as information. The DNA information code comprises three regions, i.e., primer regions located on both ends, buffer regions adjacent to the primer regions, and an information code region in the center.

Each of the primers has a length of about 20-30 base pairs and is necessary to amplify the DNA and read the DNA base sequence. If the base sequence of the primer is unknown, the DNA cannot be amplified and read. Thus, copying of the DNA information code can be prevented primarily by maintaining the base sequence of the primer region confidential.

The buffer regions are respectively interposed between the information code region and the primer region and have various buffering effects. First, when identifying the DNA base pair sequence, base sequences near the primer cannot be read, and thus, the buffer regions are required. In addition, when the buffer regions are present, the start point of the information code can be properly concealed, and thus, copying the information can be prevented. If the start point of the information code has been previously designated during production of the code and kept confidential, the information code cannot be interpreted with the unknown start point, thus copying is prevented.

The information code region has a specific sequence according to the previously designated coding method and contains the characteristics and the relevant information of the relevant medium (articles or documents, etc.).

The DNA information code prepared by arbitrarily manipulating a base pair may be encapsulated with an inorganic material, etc. to be protected from the extreme environmental factors. Especially, a layered compound, such as a layered double hydroxide (Mg2Al(OH)6(NO3)) has a cationic layer charge, and thus, may bind to DNA having negatively charged phosphate groups through electrostatic interaction. The layered double hydroxide which can stabilize the DNA between its layers through electrostatic interaction can protect the DNA especially against enzymes, such as DNase, which may fatally act on DNA, and can securely preserve the DNA at pH 3 or higher.

A layered double hydroxide (LDH) is referred to as a hydrotalcite-like compound. The layered double hydroxide refers to a compound having the structure similar to that of hydrotalcite which is a magnesium/aluminum layered double hydroxide, wherein magnesium and aluminum may be substituted with other divalent and trivalent metals, respectively. The layered double hydroxide is positively charged due to the presence of the interlayer trivalent metal ions and various anions can be introduced between the layers. Thus, according to an embodiment of the present invention, the DNA, which is negatively charged, can be introduced between the layers of the layered double hydroxide.

The layered double hydroxide may be generally synthesized by preparing a solution of at least two divalent and trivalent metal salts and titrating the solution with a basic solution. Magnesium (Mg2+), calcium (Ca2+), zinc (Zn2+), etc. may be used as the divalent metals and aluminum (Al3+), iron (Fe3+), etc. may be used as the trivalent metals. Sodium hydroxide (NaOH), ammonia (NH3), etc. may be used as the basic solution. The layered double hydroxide is formed by precipitation and the desired composition, particle shape and size of the layered double hydroxide may be obtained by controlling concentrations and ratios of the metal ions, a titration rate, a total of reaction time during the synthesis of the layered double hydroxide. The layered double hydroxide used as an in vivo injection must be small and uniform particles having the size of 300 nm or less, in order not to block capillaries and give a physical shock. In an embodiment of the present invention, as a result of the reaction of magnesium with aluminum for 24 hours, the layered double hydroxide particles having a uniform size can be obtained.

The encapsulating of the DNA with the obtained layered double hydroxide may be performed using an ion exchange reaction or a co-precipitation method. In the ion exchange reaction, ions, such as nitric acid (NO3-), chloride (Cl—) etc. between the layers of the layered double hydroxide are substituted with ionized DNA. In the co-precipitation method, anionic species is added to the mixed metal solution during titration, and thus, the anionic species is encapsulated at the time of forming the layers of the layered double hydroxide. Examples of the DNA which is introduced into the layered double hydroxide, include general DNA, which is negatively charged and a like-nucleic acid, such as peptide nucleic acid (PNA) and locked nucleic acid (LNA).

The layered double hydroxide encapsulating the DNA, i.e., DNA-inorganic hybrid complex may be represented by the following formula:

[M²⁺₁₋ₓN³⁺ₓ(OH)₂][Aⁿ⁻]_{x/n}.yH₂O

wherein

M²⁺ is a divalent metal cation selected from the group consisting of Mg²⁺, Ni²⁺, Cu²⁺, and Zn²⁺,

N³⁺ is a trivalent metal cation selected from the group consisting of Al³⁺, Fe³⁺, V³⁺, Ti³⁺, and Ga³⁺,

x is a number of 0.1-0.4,

A is an anionic DNA,

n is a charge number of the DNA, and

y is a positive number.

In the above formula, x is related to a mixing ratio of the metals and may be 0.1-0.4, preferably 0.25-0.33. If x is deviated from the range of 0.1-0.4, the DNA may not be encapsulated in the inorganic carrier of the layered double hydroxide, i.e., insertion between the layers may not be attained, and thus, the desired DNA-inorganic hybrid complex may not be easily formed.

The DNA-inorganic hybrid complex can be used in a hydrate form. A degree of hydration can be expressed using “y”, wherein “y” is a positive number. “y” may vary depending on various factors, such as humidity, etc. and be commonly used within a wide range.

The metal double-layered hydroxide encapsulating the DNA is a fine particle having the size of 100-200 nm and when it is sprayed on the relevant substance and held on its surface, the information code system can be implemented invisibly into the substance. When the layered double hydroxide is treated with an acidic buffer solution like a phosphate buffer solution, the layered double hydroxide is selectively dissolved in the buffer solution, and thus, the DNA may be extracted.

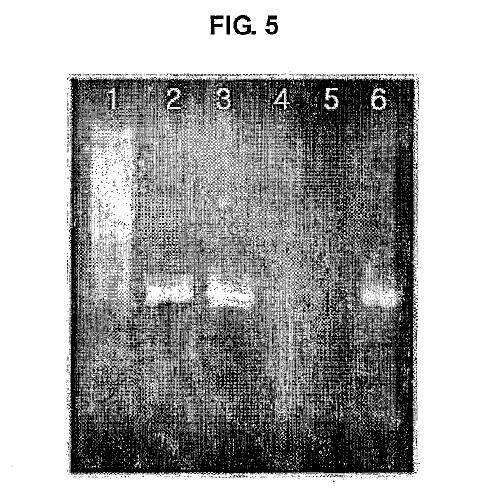

The DNA information code encapsulated with the layered double hydroxide is treated with DNase I enzyme, and then, the DNA information code is extracted from the layered double hydroxide. The extract is subjected to electrophoresis. Separately, the extract is amplified using the PCR and then subjected to electrophoresis. The DNA is not detected in the electrophoresis of the extract, since the concentration of the DNA in the extract is very low. In the electrophoresis of the DNA amplified using the PCR, a clear band of DNA is observed (see FIG. 5). Considering the fact that the PCR does not proceed when a portion of DNA is modified or destroyed, from the observed DNA band of the PCR amplified sample, it is confirmed that the PCR was efficiently carried out and the original DNA information code was preserved in the amplified sample. Thus, it is confirmed that when the DNA information code is encapsulated with the layered double hydroxide, the DNA information code can be securely preserved against the environmental factors, such as enzymes, etc.

The extracted DNA information code contains a trace amount of DNA. Thus, unless the DNA is collected using an efficient method, the DNA information cannot be read. Polypyrrole-maghemite nanohybrid, which is a hybrid material obtained by coating polypyrrole polymer, which has an ability to detect DNA, on a superparamagnetic maghemite nanoparticle having the size of about 10 nm or less, may collect the DNA using a magnet. The polypyrrole-maghemite nanohybrid ensures an easy collection of a trace amount of DNA using magnetic forces.

The obtained polypyrrole-maghemite nanohybrids are dispersed in the DNA solution to be detected, and then, the polypyrrole-maghemite nanohybrids having the DNAs adsorbed thereto are collected by the magnetic forces. The collected polypyrrole-maghemite nanohybrids are dispersed in distilled water, and then, the PCR is facilitated. A DNA sample amplified using the PCR is subjected to electrophoresis using an agarose gel, and then, a DNA band may be analyzed to detect the presence of DNA.

The polypyrrole-maghemite nanohybrids can make it possible to detect a trace amount of DNA, which cannot be detected by conventional filtering and detecting methods for a gene. The DNA adsorbed on the polypyrrole-maghemite nanohybrid can be easily separated from other impurities using magnetic forces. The adsorbed DNA can be amplified using the PCR. Thus, an ultra-low concentration of DNA, for example, at a femtomole (10-15 mol/L) level, which cannot be detected and analyzed using the conventional methods, can be collected using the polypyrrole-maghemite nanohybrids.

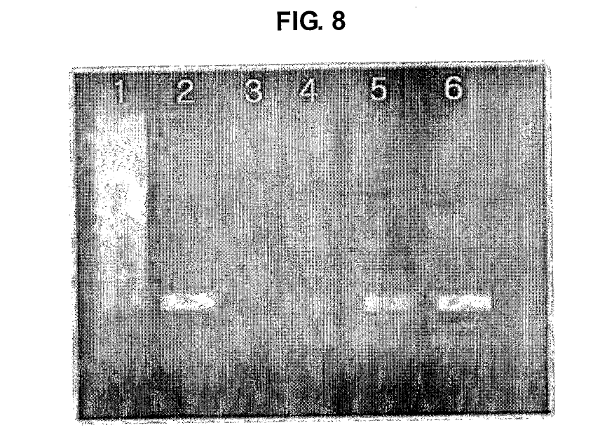

The DNA information code may be amplified using the PCR and its information may be read. From the electrophoretic results of the amplified DNA information code, it is confirmed that the amplified DNA is identical to the original DNA. Thus, it is understood that using the polypyrrole-maghemite nanohybrid, the DNA information can be collected with a low risk of damage and a low concentration of DNA can be collected to read the DNA information (see FIG. 8).

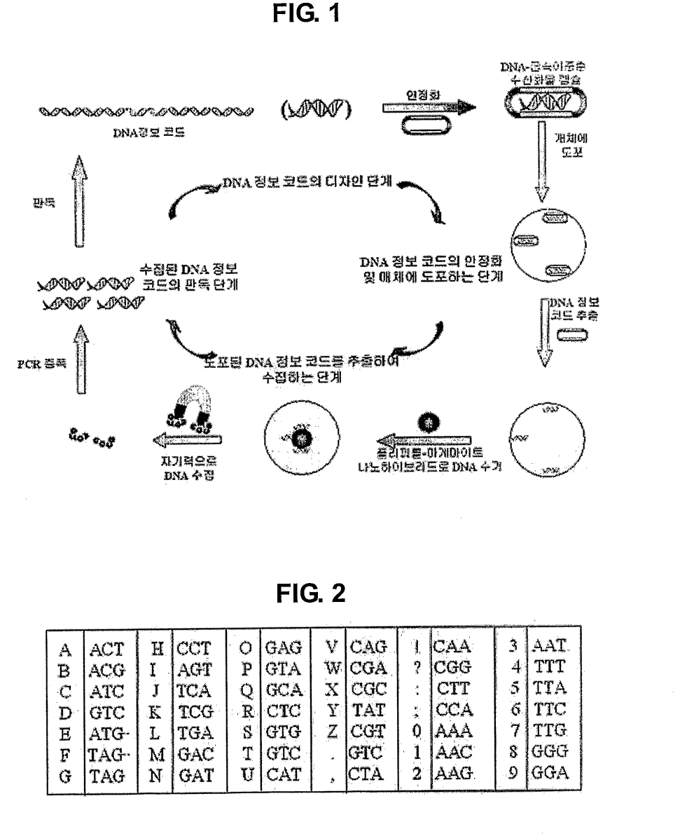

Accordingly, the present invention can establish the DNA information code system of the following four operational schemes. First, DNA information code is prepared by manipulating base pair sequence, which is unable to duplicate. Second, the DNA information code is encrypted by inserting DNA information code into the layered double hydroxide and making the DNA inert. Third, a trace amount of DNA information code is collected and concentrated using a polypyrrole-maghemite hybrid nanoparticles. Fourth, the collected DNA is amplified using the PCR and decoded using electrophoresis (see FIG. 1).

The characteristics of the DNA-layered double hydroxide capsule and polypyrrole-maghemite nanohybrid particle prepared according to embodiments of the present invention and the analysis of the DNA were estimated as follows.

1) Estimation Using X-Ray Diffraction

Pre-treatment of sample: drying in the form of solid powders

Measuring instrument: Philips

Range of diffraction angle: 20-70°

2) IR Spectrum

Pre-treatment of sample: mixing with KBr and compressing into a disc form

Measuring instrument: Bruker IFS 48

Range of frequency: 400-4000 cm⁻¹

3) Electrophoresis of the Sample Amplified Using a PCR

Pre-treatment of sample: amplifying DNA obtained as a solution or colloid in each operation, using a PCR amplifier

Electrophoresis conditions: 1% agarose gel, TBE (Tris Boric EDTA) buffer solution, at a voltage of 75 V

Hereinafter, the present invention will be described in more detail with reference to the following examples. However, these examples are given for the purpose of illustration and are not intended to limit the scope of the invention.

### EMBODIMENTS

### Example 1

A DNA information code was designed as follows. A single-stranded DNA having the length of 100 base pairs and its complementary DNA strand were separately synthesized, and then, hybridized to produce a double-stranded DNA. The DNA information code was designed such that it is composed of primers on both ends, which correspond to the 1-20^{th} base pairs and the 81-100^{th} base pairs, buffer regions which correspond to the 21-40^{th} base pairs and the 59-80^{th} base pairs, and an information code region which correspond to the 41-58^{th} base pairs. The information code region which corresponds to 41-58^{th} base pairs had a sequence of 5′-CCT TAT ACG CTC AGT GTC-3′ and was designed to designate six letters according to the three-base pairs coding method and code the word “HYBRID” according to the information code listing table illustrated in FIG. 2.

FIG. 2 is an information code listing table according to the three-base pairs coding method, which is used for substituting the information into the DNA information code according to an embodiment of the present invention. A three-base pair is substituted into a letter or a number according to the information code listing table and the information code used in Example 1 represents the word “HYBRID”.

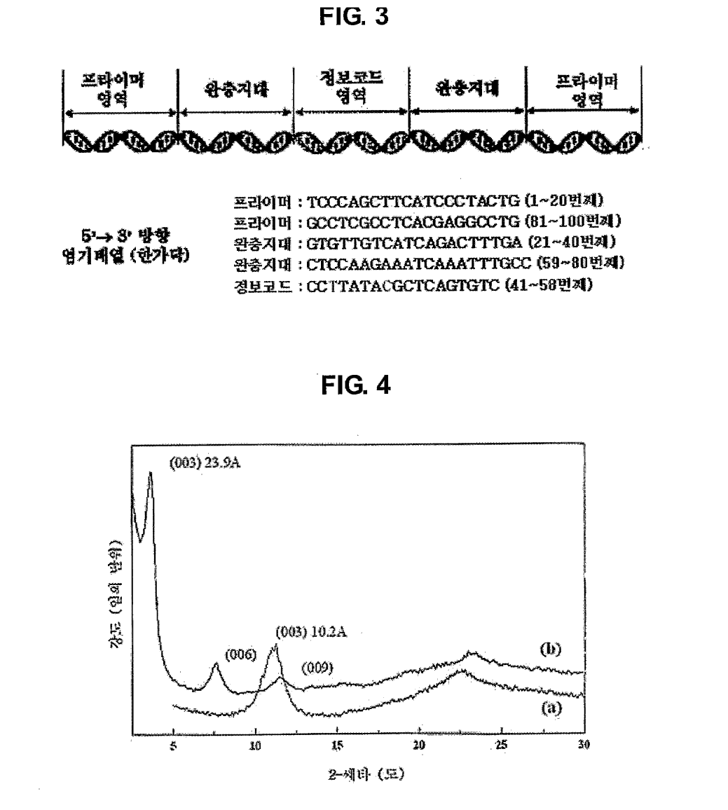

FIG. 3 is a schematic view illustrating a base pair sequence of the DNA information code used in Example 1. Referring to FIG. 3, by arranging the primers, the buffer regions, and the information code region within the DNA which has the length of 100 base pairs, the security of the information code can be maintained.

### Example 2

The DNA information code was capsulated with a layered double hydroxide to be stabilized against the environmental factors. The layered double hydroxide (Mg₂Al(OH)₆(NO₃)) was synthesized by titrating a 0.1 M aqueous solution in which magnesium nitrate and aluminum nitrate were mixed in a ratio of 2:1, with 0.1 M sodium hydroxide solution until a pH of 9.5 in a nitrogen atmosphere. The synthesized layered double hydroxide was freeze-dried to be used for encapsulating the DNA. In the encapsulation, 10 mg of the DNA and 10 mg of the layered double hydroxide were dispersed in 1 mL of decarbonated distilled water and the dispersed slurry was stirred for 7 days at 75° C. in a nitrogen atmosphere.

FIG. 4(a) represents an X-ray diffraction pattern of the layered double hydroxide used for encapsulating the DNA. FIG. 4(b) shows an X-ray diffraction pattern of DNA-layered double hydroxide hybrid, which was stabilized by encapsulating DNA by layered double hydroxide. Peak (003) corresponds to total thickness of the layers plus the interlayer distance. Insertion of DNA into the interlayer gives rise to the interlayer distance change from 10.2 Å to 23.9 Å confirming that the DNA was stably inserted between the layers of the layered double hydroxide.

### Example 3

The stability of the DNA-layered double hydroxide hybrid against the enzymatic reaction was tested by enzyme treatment. 10 mg of the DNA-layered double hydroxide hybrid was dispersed in 10 mL of distilled water and treated with 96 units of DNase I/Tris buffer solution (100 uL). Then, Ca²⁺/Mg²⁺ ions were added to the obtained product and incubated at 37° C. for 2 hours. Likely, the DNA was treated with DNase I enzyme and incubated as described above. After the incubation was completed, the resultant products were adjusted to about pH of 2.5 by adding a phosphate buffer solution and stirred for 30 minutes to dissolve the layered double hydroxide and then extract the DNA. The extracted DNA was subjected to electrophoresis with and without PCR amplification.

The PCRs were carried out using a 25 uL of 1×PCR buffer solution comprising each 200 uM of dNTP, each 0.2 uM of primer, and 1 U of Taq polymerase (Nova-taq, Genemed). The conditions of the PCR were as follows: the initial treatment at 95° C. for 10 minutes; 35 cycles with one cycle including heating at 95° C. for 30 sec, 60° C. for 30 sec, and 72° C. for 30 sec; and the final treatment at 72° C. for 10 minutes.

FIG. 5 is an electrophoresis result showing whether there is a damage to the DNA during the treatment of DNase I. Lane 1 indicates a marker DNA, which exhibits ladder-shaped DNA bands in every 100 bp. Lane 2 indicates the DNA designed in Example 1 as the control. Lane 3 indicates the results of the DNA information code which was extracted from the DNA-layered double hydroxide hybrid. Lane 4 indicates the results of the DNA information code which was obtained after treating the control of Example 1 with DNase I enzyme. Lane 5 indicates the results of the DNA, which was extracted from DNA-layered double hydroxide hybrid after treating it with DNase I enzyme. Lane 6 indicates the results of the DNA which was obtained from PCR amplification of the extract from the DNA-layered double hydroxide hybrid. Before the extraction, the DNA-layered double hydroxide hybrid was treated with DNase I.

It was confirmed from FIG. 5 that the DNA which was not encapsulated in the layered double hydroxide was completely decomposed by the enzyme, while the DNA which was encapsulated in the layered double hydroxide and thus stabilized, was maintained without being damaged or decomposed. In addition, when the extracted DNA was not PCR-amplified, no DNA band was observed in the electrophoresis since the concentration of the DNA was too low. Meanwhile, when the extracted DNA was PCR-amplified, a DNA band was observed. Thus, it was confirmed that the PCR was efficiently facilitated and that the DNA encapsulated in the layered double hydroxide was not badly damaged even after being treated with DNase I.

### Example 4

Maghemite nanoparticles were synthesized as follows. The respective aqueous solutions of divalent and trivalent iron chlorides (Fe²⁺=43.9 mM, Fe³⁺=87.8 mM) were mixed in a ratio of Fe²⁺/Fe³⁺=0.5 and titration was performed with aqueous ammonia to make the mixed solution basic. Thus, 5 g of Fe₃O₄ magnetite nanoparticles were precipitated. The precipitated magnetite was oxidized by treating it with nitric acid, and then 0.1 g of iron nitrate (Fe(NO₃)₃) was added to oxidize the surface of the precipitates. Through this process, the magnetite nanoparticles were oxidized to maghemite nanoparticles.

The synthesized maghemite nanoparticles were coated with polypyrrole to obtain polypyrrole-maghemite nanohybrid particles using the following process. 1.7 g of maghemite nanoparticles were dispersed in an excess of liquid pyrrole (a mass ratio of pyrrole/maghemite >0.7) and an excess of pyrrole was removed to obtain the maghemite nanoparticles with their surfaces wetted with pyrrole. An ethanol solution containing 0.15 M trivalent iron chloride was added to the wet maghemite nanoparticles and stirred for 30 minutes to polymerize the pyrrole. After the polymerisation, the resultant product was rinsed with ethanol to remove the unreacted pyrrole therefrom.

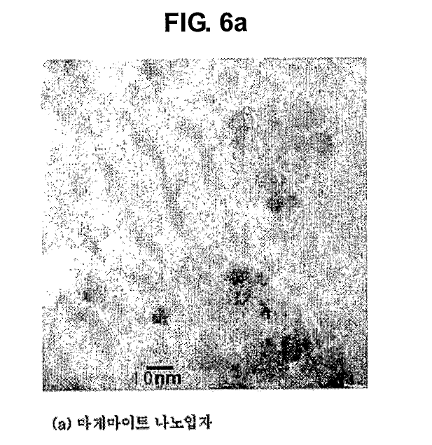

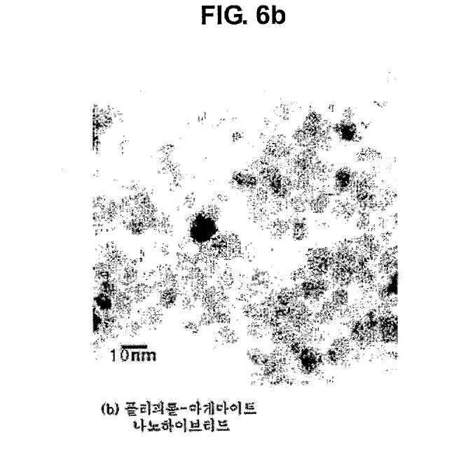

FIG. 6A is a transmission electron microscope photo of maghemite nanoparticles. In FIG. 6A, the synthesized maghemite has an average size of 7 nm. FIG. 6B is a transmission electron microscope photo of polypyrrole-maghemite nanohybrids. It was confirmed from FIG. 6B that the shape of maghemite was not changed and light grey colored-polymer regions are present between the maghemite particles, and thus, the maghemite particles were uniformly coated with polypyrrole.

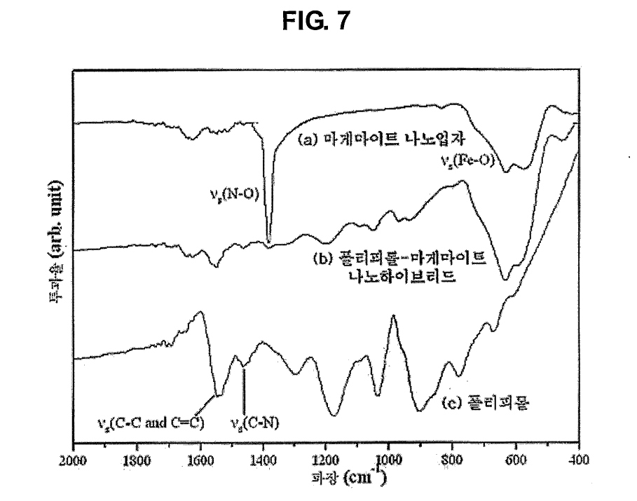

Referring to FIG. 7, (a) represents an infrared (IR) spectrum for the maghemite nanoparticles, (b) represents an IR spectrum for polypyrrole-maghemite nanohybrids, and (c) represents an IR spectrum for polypyrrole. It was confirmed from FIG. 7 that maghemite is coated with polypyrrole.

### Example 5

The DNA information code extracted from the layered double hydroxide capsule was diluted to obtain a solution having the DNA in a low concentration at a pM level or less. The DNA solution was amplified using the PCR and finally subjected to electrophoresis. Separately, the same DNA solution was mixed with the polypyrrole-maghemite nanohybrid and the DNA was extracted, amplified, and subjected to electrophoresis.

For this, first, 1 mL of 100 fM DNA solution was amplified using the PCR (forward primer: TCC CAG CTT CAT CCC TAC TG, reverse primer: CAG GCC TCG TGA GGC GAG GC, compositional ratio; template:10×PCR reaction buffer:dNTP:forward primer (10 μM):reverse primer (10 μM):100×BSA:Taq:D.W=1:2.5:2:0.5:0.5:0.25:0.2:18. PCR conditions: 30 cycles with one cycle at 95° C. for 30 sec, at 60° C. for 10 sec, and at 72° C. for 30 sec). Separately, 1 mg of polypyrrole-maghemite nanohybrid was dispersed in 1 mL of a 100 fM DNA solution at 37° C. for 6 hours. Then, the polypyrrole-maghemite portions in the dispersion were collected using a magnet and dispersed in distilled water for rinsing. The rinsing was carried out 5 times and in each rinsing, 1 mL of the supernatant in the dispersion was taken and subjected to the PCR.

After the rinsing by 5 times, 1 μl of the magnet-collected wet polypyrrole-maghemite nanohybrid was taken and subjected to the PCR. For a 500 fM DNA solution, the same procedure was performed. All the samples were subjected to electrophoresis on agarose gels and stained with ethidium bromide and irradiated with UV light to analyze the DNA bands. The determined DNA bands are shown in FIG. 8.

Referring to FIG. 8, Lane 1 indicates the results of a marker DNA, which exhibits ladder-shaped DNA bands in every 100 bp. Lane 2 indicates the results of a positive control DNA designed in Example 1. Lane 3 indicates the PCR-amplified DNA band of the 100 fM DNA solution. Lane 4 indicates the PCR-amplified DNA band of the 500 fM DNA solution. Lane 5 indicates the PCR-amplified DNA band, in which the DNA was collected by adsorbing the DNA from the 100 fM DNA solution to polypyrrole-maghemite nanohybrid. Lane 6 indicates the PCR-amplified DNA band, in which the DNA was collected by be adsorbed from the 500 fM DNA solution to polypyrrole-maghemite nanohybrid.

According to the results of the DNA analysis in FIG. 8, when the 100 fM DNA solution and the 500 fM DNA solution were amplified using the PCR, the DNA band was difficult to detect. Thus, it was confirmed that the DNA at 500 fM or less cannot be detected using only the PCR amplification. However, when the DNA was adsorbed to polypyrrole-maghemite nanohybrid, rinsed several times, and then PCR-amplified, a DNA band was clearly detected in the electrophoresis. Thus, it was confirmed that the DNA was efficiently adsorbed to the polypyrrole-maghemite nanohybrid and for detecting a trace amount of DNA, it is proper to collect the DNA using the polypyrrole-maghemite nanohybrid, and then, perform the PCR amplification.

In addition, it was confirmed that by collecting the DNA information code using the polypyrrole-maghemite nanohybrid, the DNA information can be read without being damaged. Thus, it is understood that the polypyrrole-maghemite nanohybrid can be properly used for collecting and reading the information of the molecular code system using DNA.

As explained above, according to an embodiment of the present invention, DNA having an arbitrarily manipulated base sequence is designated as a molecular level of information code and primers and buffer regions as security units may be located in the DNA. In addition, the DNA information code thus designed may be encapsulated with an inorganic material, such as a layered double hydroxide, to be protected from the environmental factors and may be coated invisible on a medium to function as a confidential information code.

The DNA information may be extracted by taking a portion of the DNA information code coated on the medium and extracting only the DNA therefrom. The efficient extraction of the DNA present in a trace amount may be performed using the polypyrrole-maghemite nanohybrid. The extracted DNA information code is collected. Then, the collected DNA information code may be efficiently amplified using the PCR and the original DNA information may be read using electrophoresis, etc. Thus, the information code system is provided at a molecular level having a high security, as suggested above.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A DNA information code comprising an information code region containing a specific base sequence, primer regions located on both ends and necessary to amplify the DNA and read the DNA base sequence, and buffer regions, each buffer region being interposed between the information code region and the primer region and separating the information code region from the primer region by at least one nucleotide.

2. The DNA information code of claim 1, wherein the base sequence of the information code region corresponds to a specific letter and/or number row.

3. The DNA information code of claim 2, wherein in the base sequence of the information code region, each three-base pair corresponds to a letter or a number.

4. The DNA information code of claim 1, wherein a sequence of the primer region is kept confidential.

5. A method of stabilizing a DNA information code, comprising:
preparing the DNA information code of claim 1; and
encapsulating the DNA information code with a layered double hydroxide.

6. The method of claim 5, wherein the layered double hydroxide having encapsulated the DNA information code therein is represented by the following formula:
[M²⁺₁₋ₓN³⁺ₓ(OH)₂][Aⁿ⁻]_{x/n}.yH₂O
wherein
M²⁺ is a divalent metal cation selected from the group consisting of Mg²⁺, Ni²⁺, Cu²⁺, and Zn²⁺,
N³⁺ is a trivalent metal cation selected from the group consisting of Al³⁺, Fe³⁺, V³⁺, Ti³⁺, and Ga³⁺,
x is a number of 0.1-0.4,
A is an anionic DNA,
n is a charge number of the DNA, and
y is a positive number.

7. The method of claim 6, wherein the layered double hydroxide is Mg₂Al(OH)₆(NO₃).

8. The method of claim 7, wherein the layered double hydroxide is synthesized by titrating a 0.01-0.5 M aqueous solution in which magnesium nitrate and aluminum nitrate are mixed in a ratio of 1.5:1-2.5:1, with a 0.01-0.5 M sodium hydroxide solution until a pH of 9-10 under a nitrogen atmosphere.

9. The method of claim 5, wherein the encapsulating comprises dispersing the DNA information code and the layered double hydroxide in a molar ratio of 1:1-2:1 in decarbonated distilled water and stirring the obtained dispersion at 65-75° C. for 5-14 days under a nitrogen atmosphere.

10. A DNA information code system which is stabilized using the method of claim 5.

11. The DNA information code system of claim 10, which is bound to a medium.

12. A method of detecting a specific DNA information code from a medium coated with the DNA information code system of claim 10, comprising:
taking the DNA-layered double hydroxide capsule from the medium;
extracting the DNA information code by dissolving the layered double hydroxide in a solvent;
collecting the extracted DNA information code using polypyrrole-maghemite hybrid nanoparticles;
amplifying the collected DNA information code using a polymerase chain reaction (PCR); and
reading the amplified DNA information codes.

13. The method of claim 12, wherein the extracting the DNA information code comprises dispersing the DNA-layered double hydroxide capsule in distilled water, adjusting the pH of the resultant dispersion to 2.5-3 by adding a phosphate buffer solution, and then, stirring the dispersion for 20-40 minutes to dissolve the layered double hydroxide layer.

14. The method of claim 12, wherein in the collecting the extracted DNA information code, a concentration of the DNA is 500 femtomole (10⁻¹⁵ mol/L) or less.

15. The method of claim 14, wherein the concentration of the DNA is 100 femtomole or less.

16. The method of claim 12, wherein in the reading the amplified DNA information code, whether the amplified DNA information code is identical to a predetermined DNA information code is read using electrophoresis.

17. The method of claim 12, wherein the reading the amplified DNA information code comprises sequencing the amplified DNA using an automatic sequencer and converting the sequence to a corresponding letter and/or number row.

18. A method of collecting a DNA information code extracted from a DNA-layered double hydroxide using polypyrrole-maghemite hybrid nanoparticles.

19. The method of claim 18, wherein the polypyrrole-maghemite hybrid nanoparticles are synthesized by dispersing maghemite nanoparticles in an excess of liquid pyrrole, removing an excess of pyrrole to obtain the maghemite nanoparticles with their surfaces wetted with pyrrole, adding an ethanol solution containing 0.1-0.2 M trivalent iron chloride to the wet maghemite nanoparticles and stirring for 30 minutes to 1 hour to polymerize the pyrrole, and then, rinsing the resultant product with ethanol to remove an unreacted pyrrole therefrom.

20. The method of claim 18, wherein the polypyrrole-maghemite hybrid nanoparticles are mixed with the extracted DNA solution to obtain a dispersion, and then, polypyrrole-maghemite portions in the dispersion are collected using a magnet.
